(19) United States
(12) Patent Application Publication
(10) Pub. No.: US 2010/0074927 A1
(43) Pub. Date: Mar. 25, 2010

(54) DELIVERY OF THERAPEUTIC COMPOUNDS VIA MICROPARTICLES OR MICROBUBBLES

(76) Inventors: Patrick L. Iversen, Corvallis, OR (US); Nicholas Kipshidze, New York, NY (US)

Correspondence Address: SEED INTELLECTUAL PROPERTY LAW GROUP PLLC, 701 FIFTH AVE, SUITE 5400, SEATTLE, WA, 98104, US

(21) Appl. No.: 12/561,991

(22) Filed: Sep. 17, 2009

Related U.S. Application Data
(63) Continuation of application No. 10/668,988, filed on Sep. 22, 2003, now abandoned.
Application No. 10/668,988 is a continuation-in-part of application No. 10/190,419, filed on Jul. 2, 2002.
Application No. 10/190,419 is a continuation-in-part of application No. 10/138,589, filed on May 3, 2002, now abandoned.

(51) Int. Cl. A61K 9/00; A61K 33/24; A61K 31/7048; A61K 31/704; A61K 31/7068; A61K 31/675; A61K 31/519; A61K 31/505; A61K 31/436; A61K 31/4375; A61K 31/4745; A61K 31/337; A61K 31/282; A61K 31/166; A61K 31/138; A61P 35/00

(52) U.S. Cl. 424/400; 424/649; 514/27; 514/34; 514/49; 514/90; 514/249; 514/274; 514/291; 514/283; 514/449; 514/492; 514/588; 514/615; 514/648

(57) ABSTRACT

Microparticle carriers, particularly protein-encapsulated microbubbles, are used to deliver antiproliferative drugs to target sites in a subject. In particular, antirestenotic drugs are delivered to areas of vascular injury for treatment or prevention of hyperproliferative disease, e.g. stenosis, in blood vessels; and antineoplastic drugs are targeted to tumor sites.

### CROSS-REFERENCE TO RELATED APPLICATIONS

This application is a continuation of U.S. Ser. No. 10/668,988, filed Sep. 22, 2003, which is a continuation-in-part of U.S. Ser. No. 10/190,419, filed Jul. 2, 2002, which is a continuation-in-part of U.S. Ser. No. 10/138,589, filed May 3, 2002. Each of these applications is incorporated herein in its entirety by reference.

### FIELD OF THE INVENTION

The present invention relates to methods and compositions for delivery of antiproliferative drugs to particular target sites. In particular, antirestenotic drugs are delivered to areas of vascular injury for treatment or prevention of hyperproliferative disease, e.g. stenosis, in blood vessels, and antineoplastic drugs are targeted to tumor sites.

### REFERENCE

Barbarese E et al., J. Neuro-Oncology 26:25-34 (October 1995).

Casterella P J et al., Cardiol Rev, July-August 1999, 7(4):219-31.

Cleland J L, Biotech Progress, January-February 1998, 14(1):102-7.

D'Arrigo J S et al., Investigative Radiology 28(3):218-222 (1993).

D'Arrigo J S et J. Neuroimag. 1:134-139 (1991).

Herrmann S M et al., “Polymorphisms of the human matrix gla-protein gene (MGP); vascular calcification and myocardial infarction.” Arterioscler Thromb Vasc Biol 2000; 20:2836-93.

Ho S et al., Neurosurgery 40(6):1260-1268 (June 1997).

Iversen P and Weller D, PCT Pubn. No. WO 00/44897, “Method of Treating Restenosis by Antisense Targeting of c-myc.” (August 3 2000).

Kipshidze N et al., Catheter Cardvac. Interv. 54(2):247-56 (October 2001).

Kornowski R, Hong M K, Tio F O et al., “In-stent restenosis: contributions of inflammatory responses and arterial injury to neointimal hyperplasia.” J Am Coll Cardiol 1998; 31:224-230.

Kreuter J, J Anatomy, December 1996, 189(Pt 3):503-5.

Kwon G S, Crit Rev In Therap Drug Carrier Systems 1998, 15(5):481-512.

Lindler J R et al., Echocardiography 18(4):329-337 (May 2001).

Lindler J R et al., J. Am. Coll. Cardiol. 33(2 Suppl A):407A-408A (February 1999).

Lindler J R, Am. J. Cardiol. 90(Suppl):72J-80J (November 2002).

Porter T R et al., J Ultrasound Med, August 1996, 15(8):577.

Quintanar-Guerrero D et al., Drug Dev Ind Pharm December 1998, 24(12):1113-28.

Ravi Kumar M N, J Pharm & Pharm Sci May-August 2000, 3(2):234-58.

Simon R H et al., Ultrasound in Medicine & Biology 19(2):123-125 (1993).

Soppimath, K S et al., J Controlled Release Jan. 29, 2001, 70(1-2):1-20.

### BACKGROUND OF THE INVENTION

Drug delivery techniques are continually being developed in drug therapy to control, regulate, and target the release of drugs in the body. Goals include augmentation of drug availability, maintenance of constant and continuous therapeutic levels of a drug in the systemic circulation or at a specific target organ site, reduction of dosages and/or frequency of administration required to realize the desired therapeutic benefit, and consequent reduction of drug-induced side effects. Drug delivery systems currently include, for example, carriers based on proteins, polysaccharides, synthetic polymers, and liposomes.

Gas filled microbubbles have been conventionally used as contrast agents for diagnostic ultrasound. They have also been described for therapeutic applications, such as enhancement of drug penetration (Tachibana et al., U.S. Pat. No. 5,315,998), as thrombolytics (e.g. Porter, U.S. Pat. No. 5,648,098), and for drug delivery. Reports of use of microbubbles for drug delivery have generally described the use of some external method of releasing the drug from the microbubbles at the site of delivery, by, for example, raising the temperature to induce a phase change (Unger, U.S. Pat. No. 6,143,276) or exposing the microbubbles to ultrasound (Unger, U.S. Pat. No. 6,143,276; Klaveness et al., U.S. Pat. No. 6,261,537; Lindler et al., Echocardiography 18(4):329, May 2001, and Unger et al., Echocardiography 18(4):355, May 2001; Porter et al., U.S. Pat. No. 6,117,858).

As described in co-owned U.S. Pat. No. 5,849,727, the applicant showed that gas filled, protein-encapsulated microbubbles, conventionally employed as contrast agents in ultrasonic imaging, could be conjugated to therapeutic agents. As described therein, while release of the agent at a target site may comprise the use of ultrasound, the use of ultrasound is not a requirement.

### SUMMARY OF THE INVENTION

In one aspect, the present invention provides a composition comprising an antiproliferative therapeutic agent and a suspension of microbubbles, which are encapsulated with a filmogenic fluid and contain a gas selected from a perfluorocarbon and SF₆. Such a composition is generally formed by incubating the antiproliferative agent of choice with a suspension of microbubbles, and is provided in isolated form for administration. The gas contained within the microbubbles is preferably a perfluorocarbon and is preferably selected from the group consisting of perfluoromethane, perfluoroethane, perfluoropropane, perfluorobutane, and perfluoropentane. Perfluorobutane and perfluoropentane and particularly preferred.

The filmogenic fluid encapsulating the microbubbles is preferably selected from the group consisting of proteins, surfactants, polysaccharides, and combinations thereof, and more preferably is selected from a filmogenic protein, a polysaccharide, and combinations thereof. In one embodiment, the fluid comprises a filmogenic protein, such as human serum albumin. The protein may be provided as a mixture with a polysaccharide such as dextrose.

In selected embodiments, the antiproliferative agent is selected from the group consisting of rapamycin, tacrolimus, paclitaxel, other taxanes, such as docetaxel, and active analogs, derivatives or prodrugs of these compounds. Preferably, the antiproliferative agent is a non-antisense agent. In selected embodiments, the agent is not an oligonucleotide or oligonucleotide analog.

In further embodiments, the antiproliferative agent is selected from the group consisting of cisplatin, carboplatin, etoposide, tamoxifen, methotrexate, 5-fluorouracil, adriamycin, daunorubicin, doxorubicin, vincristine, and vinblastine. In still further embodiments, the antiproliferative agent is selected from the group consisting of cisplatin, carboplatin, methotrexate, 5-fluorouracil, vincristine, and vinblastine. In other selected embodiments, the antiproliferative agent is selected from the group consisting of amsacrine, mitotane, topotecan, tretinoin, hydroxyurea, procarbazine, carmustine, mechlorethamine hydrochloride, cyclophosphamide, ifosfamide, chlorambucil, melphalan, busulfan, thiotepa, carmustine, estramustine, dacarbazine, omustine, streptozocin, vincristine, vinblastine, vinorelbine, vindesine, fludarabine, fluorodeoxyuridine, cytosine arabinoside, cytarabine, azidothymidine, cysteine arabinoside, azacytidine, mercaptopurine, thioguanine, cladribine, pentostatin, arabinosyl adenine, dactinomycin, daunorubicin, doxorubicin, amsacrine, idarubicin, mitoxantrone, bleomycin, plicamycin, ansamitomycin, mitomycin, aminoglutethimide, and flutamide.

In another aspect, the present invention provides a method for delivering an antiproliferative therapeutic agent to a tumor site in a subject. The agent is delivered by administering parenterally to the subject a composition as described above comprising the antiproliferative therapeutic agent and a suspension of microbubbles. Preferably, the antiproliferative therapeutic agent is selected from those listed above.

The subject is preferably a mammalian subject, such as a human subject or patient. The composition of suspended microbubble/agent conjugate is administered internally to the subject, preferably parenterally, e.g. intravenously, percutaneously, intraperitoneally, intramuscularly, or intrathecally. The microbubble carrier delivers the agent or agents to the target site, where, in a preferred embodiment, the agent is released without the use of external stimulation. However, if desired, release of the agent may be modulated by application of a stimulus such as radiation, heat, or ultrasound. Application of such a stimulus may also be used to convert a prodrug to the active form of the drug, which is then released.

### DETAILED DESCRIPTION OF THE INVENTION

### I. Carrier Compositions

The present therapeutic compositions comprise a drug which is conjugated to a microparticle carrier, such as a gaseous microbubble in a fluid medium or a polymeric microparticle, with sufficient stability that the drug can be carried to and released at a site of vascular injury in a subject. Such conjugation typically refers to noncovalent binding or other association of the drug with the particle, and may be brought about by incubation with a microbubble suspension, as described further below, or intimate mixing of the drug with a polymeric microparticle carrier. A “site of vascular injury” or “site of trauma” may be defined as any region of the vessel subjected to excessive pressure, incision, abrasion, or radiation, or other phenomena which would, in the absence of treatment, tend to result in the development of stenosis. Such sites are typically characterized by the presence of damaged vascular endothelium.

In one embodiment, the pharmaceutical composition comprises a liquid suspension, preferably an aqueous suspension, of microbubbles containing a blood-insoluble gas. The microbubbles are preferably about 0.1 to 10μ in diameter. Generally, any blood-insoluble gas which is nontoxic and gaseous at body temperature can be used. The insoluble gas should have a diffusion coefficient and blood solubility lower than nitrogen or oxygen, which diffuse in the internal atmosphere of the blood vessel. Examples of useful gases are the noble gases, e.g. helium or argon, as well as fluorocarbon gases and sulfur hexafluoride. Generally, perfluorocarbon gases, such as perfluoromethane, perfluoroethane, perfluoropropane, perfluorobutane, and perfluoropentane, are preferred. It is believed that the cell membrane fluidizing feature of the perfluorobutane gas enhances cell entry for drugs on the surface of bubbles that come into contact with denuded vessel surfaces, as described further below.

The gaseous microbubbles are stabilized by a fluid filmogenic coating, to prevent coalescence and to provide an interface for binding of molecules to the microbubbles. The fluid is preferably an aqueous solution or suspension of one or more components selected from proteins, surfactants, and polysaccharides. In preferred embodiments, the components are selected from proteins, surfactant compounds, and polysaccharides. Suitable proteins include, for example, albumin, gamma globulin, apotransferrin, hemoglobin, collagen, and urease. Human proteins, e.g. human serum albumin (HSA), are preferred. In one embodiment, as described below, a mixture of HSA and dextrose is used.

Conventional surfactants include compounds such as alkyl polyether alcohols, alkylphenol polyether alcohols, and alcohol ethoxylates, having higher alkyl (e.g. 6-20 carbon atom) groups, fatty acid alkanolamides or alkylene oxide adducts thereof, and fatty acid glycerol monoesters. Surfactants particularly intended for use in microbubble contrast agent compositions are disclosed, for example, in Nycomed Imaging patents U.S. Pat. No. 6,274,120 (fatty acids, polyhydroxyalkyl esters such as esters of pentaerythritol, ethylene glycol or glycerol, fatty alcohols and amines, and esters or amides thereof, lipophilic to aldehydes and ketones; lipophilic derivatives of sugars, etc.), U.S. Pat. No. 5,990,263 (methoxy-terminated PEG acylated with e.g. 6-hexadecanoyloxyhexadecanoyl), and U.S. Pat. No. 5,919,434.

Other filmogenic synthetic polymers may also be used; see, for example, U.S. Pat. No. 6,068,857 (Weitschies) and No. 6,143,276 (Unger), which describe microbubbles having a biodegradable polymer shell, where the polymer is selected from e.g. polylactic acid, an acrylate polymer, polyacrylamide, polycyanoacrylate, a polyester, polyether, polyamide, polysiloxane, polycarbonate, or polyphosphazene, and various combinations of copolymers thereof, such as a lactic acid-glycolic acid copolymer.

Such compositions have been used as contrast agents for diagnostic ultrasound, and have also been described for therapeutic applications, such as enhancement of drug penetration (Tachibana et al., U.S. Pat. No. 5,315,998), as thrombolytics (Porter, U.S. Pat. No. 5,648,098), and for drug delivery (see below). The latter reports require some external method of releasing the drug at the site of delivery, typically by raising the temperature to induce a phase change (Unger, U.S. Pat. No. 6,143,276) or by exposing the microbubbles to ultrasound (Unger, U.S. Pat. No. 6,143,276; Klaveness et al., U.S. Pat. No. 6,261,537; Lindler et al., cited below, Unger et al., cited below; Porter et al., U.S. Pat. No. 6,117,858).

In one embodiment, the carrier is a suspension of perfluorocarbon-containing dextrose/albumin microbubbles known as PESDA (perfluorocarbon-exposed sonicated dextrose/albumin). Human serum albumin (HSA) is easily metabolized within the body and has been widely used as a contrast agent. The composition may be prepared as described in co-owned U.S. Pat. Nos. 5,849,727 and 6,117,858. Briefly, a dextrose/albumin solution is sonicated while being perfused with the perfluorocarbon gas. The microbubbles are preferably formed in an N₂-depleted, preferably N₂-free, environment, typically by introducing an N₂-depleted (in comparison to room air) or N₂-free gas into the interface between the sonicating horn and the solution. Microbubbles formed in this way are found to be significantly smaller and stabler than those formed in the presence of room air. (See e.g. Porter et al., U.S. Pat. No. 6,245,747, which is incorporated by reference).

The microbubbles are conjugated with the therapeutic agent, as described below for rapamycin. Generally, the microbubble suspension is incubated, with agitation if necessary, with a liquid formulation of the drug, such that the drug non-covalently binds at the gas/fluid interface of the microbubbles. Preferably, the liquid formulation of the drug(s) is first filtered through a micropore filter and/or sterilized. The incubation may be carried out at room temperature, or at moderately higher temperatures, as long as the stability of the drug or the microbubbles is not compromised. The microbubble/therapeutic agent composition is thus provided in isolated form for administration to a subject.

Drugs with limited aqueous solubility (such as rapamycin, tacrolimus, and paclitaxel) can be solubilized or intimately dispersed in pharmaceutically acceptable vehicles by methods known in the pharmaceutical arts. For example, rapamycin can be dissolved in, for example, alcohol, DMSO, or an oil such as castor oil or Cremophor™. A liquid formulation of rapamycin is also available from Wyeth Ayerst Pharmaceuticals, and can be used, preferably after sterilization with gamma radiation. Other solubilizing formulations are known in the art; see, for example, U.S. Pat. No. 6,267,985 (Chen and Patel, 2001), which discloses formulations containing triglycerides. and a combination of surfactants.

Other microbubble-therapeutic compositions are described in, for example, U.S. Pat. No. 6,143,276 (Unger) and No. 6,261,537 (Klaveness et al.), which are incorporated herein by reference. These references, as well as Lindler et al., Echocardiography 18(4):329, May 2001, and Unger et al., Echocardiography 18(4):355, May 2001, describe use of the microbubbles for therapeutic delivery of the conjugated compounds, in which the compounds are released from the microbubbles by application of ultrasound at the desired point of release. As described herein, neither ultrasound, nor other external stimulation, was required for delivery of therapeutically effective amounts of rapamycin to damaged endothelium in angioplasty-injured coronary vessels.

In addition to gas-filled microbubbles, other microparticles, such as biocompatible polymeric particles, may be used for delivery of a conjugated drug, e.g. rapamycin, to damaged endothelium, since very small particles tend to adhere to denuded vessel surfaces (i.e. vessels having damaged endothelium).

In this sense, “nanoparticles” refers to polymeric particles in the nanometer size range (e.g. 50 to 750 nm), while “microparticles” refers to particles in the micrometer size range (e.g. 1 to 50μ), but may also include particles in the submicromolar range, down to about 0.1μ. For use in the methods described herein, a size range of about 0.1 to 10μ is preferred. Such polymeric particles have been described for use as drug carriers into which drugs or antigens may be incorporated in the form of solid solutions or solid dispersions, or onto which these materials may be absorbed or chemically bound. See e.g. Kreuter 1996; Ravi Kumar 2000; Kwon 1998. Methods for their preparation include emulsification evaporation, solvent displacement, “salting-out”, and emulsification diffusion (Soppimath et al.; Quintanar-Guerrero et al.), as well as direct polymerization (Douglas et al.) and solvent evaporation processes (Cleland).

Preferably, the polymer is bioerodible in vivo. Biocompatible and bioerodible polymers that have been used in the art include poly(lactide-co-glycolide) copolymers, polyanhydrides, and poly(phosphoesters). Poly(orthoester) polymers designed for drug delivery, available from A.P. Pharma, Inc., are described in Heller et al., J. Controlled Release 78(1-3):133-141 (2002). In one embodiment, the polymer is a diol—diol monoglycolide—orthoester copolymer. The polymer can be produced in powdered form, e.g. by cryogrinding or spray drying, intimately mixed in powdered form with a therapeutic compound, and fabricated into various forms, including microspheres and nanospheres.

### II. Antitumor Compositions

For microbubble compositions used for delivery to a tumor site, the antiproliferative therapeutic agent to be delivered is a neoplastic agent. Known neoplastic agents include, for example, cisplatin, carboplatin, spiroplatin, iproplatin, paclitaxel, docetaxel, rapamycin, tacrolimus, asparaginase, etoposide, teniposide, tamoxifen, amsacrine, mitotane, topotecan, tretinoin, hydroxyurea, procarbazine, BCNU (carmustine) and other nitrosourea compounds, as well as others classified as alkylating agents (e.g., mechlorethamine hydrochloride, cyclophosphamide, ifosfamide, chlorambucil, melphalan, busulfan, thiotepa, carmustine, estramustine, dacarbazine, omustine, streptozocin), plant alkaloids (e.g., vincristine, vinblastine, vinorelbine, vindesine), antimetabolites (e.g., folic acid analogs, methotrexate, fludarabine), pyrimidine analogs (fluorouracil, fluorodeoxyuridine, cytosine arabinoside, cytarabine, azidothymidine, cysteine arabinoside, and azacytidine), purine analogs (mercaptopurine, thioguanine, cladribine, pentostatin, arabinosyl adenine), and antitumor antibiotics (e.g., adriamycin, dactinomycin, daunorubicin, doxorubicin, amsacrine, idarubicin, mitoxantrone, bleomycin, plicamycin, ansamitomycin, mitomycin). Also included are aminoglutethimide (an aromatase inhibitor), flutamide (an anti-androgen), gemtuzumab ozogamicin (a monoclonal antibody), and oprelvekin (a synthetic interleukin), as well as cell cycle inhibitors and EGF receptor kinase inhibitors in general.

In selected embodiments, the antiproliferative agent is selected from the group consisting of rapamycin, paclitaxel, other taxanes, such as docetaxel, and active analogs, derivatives or prodrugs of these compounds. In one embodiment, the agent is rapamycin. Preferably, the antiproliferative agent is not an antisense agent. In selected embodiments, the agent is not an oligonucleotide or oligonucleotide analog.

In further embodiments, the antiproliferative agent is selected from the group consisting of cisplatin, carboplatin, etoposide, tamoxifen, methotrexate, 5-fluorouracil, adriamycin, daunorubicin, doxorubicin, vincristine, and vinblastine. In still further embodiments, the antiproliferative agent is selected from the group consisting of cisplatin, carboplatin, methotrexate, 5-fluorouracil, vincristine, and vinblastine.

In particular, chemotherapeutic agents currently in widespread use include the platinum-containing agents, such as cisplatin and carboplatin, paclitaxel (Taxol®) and related drugs, such as docetaxel (Taxotere®), etoposide, and 5-fluorouracil. Taxol® (paclitaxel) constitutes one of the most potent drugs in cancer chemotherapy and is widely used in therapy for ovarian, breast and lung cancers. Etoposide is currently used in therapy for a variety of cancers, including testicular cancer, lung cancer, lymphoma, neuroblastoma, non-Hodgkin's lymphoma, Kaposi's Sarcoma, Wilms' Tumor, various types of leukemia, and others. Fluorouracil has been used for chemotherapy for a variety of cancers, including colon cancer, rectal cancer, breast cancer, stomach cancer, pancreatic cancer, ovarian cancer, cervical cancer, and bladder cancer.

The clinical utility of such drugs has often been limited by cost, dose-limiting adverse effects, and, in some case, such as paclitaxel, low aqueous solubility. Solubilizers such as Cremophor® (polyethoxylated castor oil) and alcohol have been demonstrated to improve solubility. Dose-limiting side effects of such drugs typically include reduction in white and red blood cell counts, nausea, loss of appetite, hair loss, joint and muscle pain, and diarrhea. By targeting the composition to the tumor site, systemic adverse effects can be reduced.

As described above, the isolated microbubble compositions are generally prepared by incubating an antiproliferative agent of choice with a suspension of microbubbles. Preferably, the microbubbles are coated with a filmogenic protein, such as albumin (or an albumin/dextrose mixture) and contain a perfluorocarbon gas, preferably perfluoropropane or perfluorobutane.

Tumors to be targeted will generally be solid tumors, which can be located anywhere in the body. Tumors for which the present delivery method is useful, include, for example, solid tumors of the brain, liver, kidney, pancreas, pituitary, colon, breast, lung, ovary, cervix, prostate, testicle, esophagus, stomach, head or neck, bone, or central nervous system. The compositions are typically administered parenterally, for example by intravenous injection or slow intravenous infusion. For localized lesions, the compositions can be administered by local injection. Intraperitoneal infusion can also be employed.

### III. Antirestenotic Compositions

For antirestenotic treatment, the therapeutic compositions include at least one immunosuppressive, antiinflammatory and/or antiproliferative drug, conjugated to and delivered by a carrier composition as described above. Examples of drugs with significant antiproliferative effects include rapamycin, paclitaxel, other taxanes, tacrolimus, angiopeptin, flavoperidol, actinomycin D, and active analogs, derivatives or prodrugs of these compounds.

Other therapeutic agents that may be used beneficially include antiinflammatory compounds, such as dexamethasone and other steroids; vassenoids; hormones such as estrogen; matrix metalloprotienase inhibitors; protease inhibitors; lipid lowering compounds; ribozymes; vascular, bone marrow and stem cells; diltiazem; acridine; clopidogrel; antithrombins; anticoagulants, such as heparin or hirudin; antioxidants; antiplatelets, such as aspirin, haloftiginore, or IIBIIIA antagonists; antibiotics; calcium channel blockers; converting enzyme inhibitors; cytokine inhibitors; growth factors; growth factor inhibitors; growth factor sequestering agents; tissue factor inhibitors; smooth muscle inhibitors; organoselenium compounds; retinoic acid and other retinoid compounds; sulfated proteoglycans; superoxide dismutase mimics; NO; NO precursors; and combinations thereof.

Synthetic glucocorticoids such as dexamethasone decrease the inflammatory response to vessel injury and may eventually decrease the restenotic process. Also useful are agents that inhibit collagen accumulation and/or calcification of the vascular wall. For example, local delivery of Vitamin K has been reported to counteract the calcification effect associated with vessel injury (Herrmann et al., 2000). Agents believed to function via different “antirestenotic mechanisms” may be expected to act synergistically. It may be useful, therefore, to combine two or more of these agents; e.g. to combine an antiproliferative and/or immunosuppressive agent with an antiinflammatory and/or an anticalcification agent.

In selected embodiments, the therapeutic agent conjugated to the microparticles is rapamycin (sirolimus), tacrolimus (FK506), paclitaxel (Taxol), epothilone D, fractionated or unfractionated heparin, or flavoperidol, or an active analog, derivative, or prodrugs of such a compound. In further embodiments, it is selected from the group consisting of rapamycin, tacrolimus, and paclitaxel, as well as active analogs or derivatives, such as prodrugs, of these compounds.

Restenosis refers to the renarrowing of the vascular lumen following vascular intervention, such as coronary artery balloon angioplasty with or without stent insertion. It is clinically defined as greater than 50% loss of initial luminal diameter gain following the procedure. Stenosis can also occur after a coronary artery bypass operation, wherein heart surgery is done to reroute, or “bypass,” blood around clogged arteries and improve the supply of blood and oxygen to the heart. In such cases, the stenosis may occur in the transplanted blood vessel segments, and particularly at the junction of replaced vessels. As noted above, stenosis can also occur at anastomotic junctions created for dialysis.

In one aspect, the invention is directed to methods for reducing the risk (incidence) or severity (extent) of stenosis, particularly following balloon angioplasty and/or stent implantation, or in response to other vessel trauma, such as following an arterial bypass operation or hemodialysis. More generally, the invention comprises methods to prevent, suppress, or treat hyperproliferative vascular disease. These methods include administering to the affected site, the above-described microbubble- or microparticle-conjugated therapeutic agent(s), in an amount effective to reduce the risk and/or severity of hyperproliferative disease. Administration may take place before, during, and/or after the procedure in question, and multiple treatments may be used. The administration may be via a route such as systemic i.v., systemic intraarterial, intracoronary, e.g. via infusion catheter, or intramural, i.e. directly to the vessel wall. When the therapeutic agent is rapamycin, preferred doses are typically between about 0.05-20 mg/kg, more preferably about 0.1 to 5.0 mg/kg. In another preferred embodiment, about 50-400 mg rapamycin per cm² of affected area is administered.

The therapeutic agents are conjugated to the microparticle carrier, preferably a microbubble composition, alone or in combination. The carrier delivers the agent or agents to the site of vessel damage, where, in a preferred embodiment, the agent is released without the use of external stimulation. As described below, delivery of rapamycin to a site of vessel injury via microbubbles did not require the use of external ultrasound, nor did it rely on a phase change in the microbubble fluid, as has been described in the prior art. However, if desired, release of the agent may also be modulated by application of a stimulus such as light, temperature variation, pressure, ultrasound or ionizing energy or magnetic field. Application of such a stimulus may also be used to convert a prodrug to the active form of the drug, which is then released.

Delivery of the compound via the above-described microparticles is effective to achieve high localized concentration of the compound at the vessel injury site, by virtue of adherence of the microparticles to damaged endothelium. By delivering drug to sites with incomplete endothelial lining, the method should be effective to treat small or branching vessels inaccessible by conventional routes, in addition to treating beyond the boundaries of coated stents.

Delivery of an antirestenotic compound, as described herein, via the above-described microparticles is advantageously used in combination with stent implantation and/or brachytherapy, since the compositions of the invention extend treatment beyond the boundaries of the stent. Microparticle delivery of the drug before treatment, immediately after treatment, or later in time can prevent or reduce the complications described above and greatly improve results obtained from implantation of a drug-eluting (or radiation-emitting) stent.

IV. In vivo Restenosis Treatment Studies

As shown below, rapamycin conjugated to PESDA and administered intravenously showed evidence of penetration into damaged vessels four hours after balloon angioplasty and administration of the composition, and significantly reduced arterial stenosis, in comparison to a control group and a c-myc antisense treated group.

In the study, seven immature farm pigs were divided into acute and chronic treatment groups. The two acute animals were treated with balloon angioplasty followed by implantation of stents in three separate coronary vessels. One received PESDA microbubbles with rapamycin (2 mg total dose) adsorbed, and the other received PESDA microbubbles with an antisense c-myc agent adsorbed. The antisense agent was a phosphorodiamidate-linked morpholino oligomer (see e.g. Summerton and Weller, Antisense Nucleic Acid Drug Dev. 7:63-70, 1997) having a sequence targeted to the ATG translation site of c-myc mRNA (see e.g. Iversen and Weller, PCT Pubn. No. WO 00/44897).

A. Acute Effects

The pigs were sacrificed four hours after treatment, and vessel tissue was examined for expression of p21, p27, β-actin and c-myc. Rapamycin enhances the expression of p21 and p27 and should have no effect on β-actin. The antisense c-myc should inhibit the expression of myc, with no effect on β-actin and minimal effect on p21 or p27. Hence, administration of c-myc antisense represents a control for rapamycin treatment, and the rapamycin represents a control for c-myc antisense agent.

Western blot analysis of p21, p27 and β-actin expression was determined by densitometry of bands appearing at the appropriate molecular weight. The band density of p21 relative to β-actin and p27 relative to β-actin are provided in the table below: (LCX=left circumflex artery; LAD=left anterior descending; RCA=right coronary artery)

TABLE 1

| | p21/β-actin ratio | | p27/β-actin ratio | |
|---|---|---|---|---|
| Vessel | Rap/PESDA | PMO/PESDA | Rap/PESDA | PMO/PESDA |
| LCX | 0.714 | 0.221 | 1.251 | 0.421 |
| LAD | 1.001 | 0.229 | 3.348 | 1.864 |
| RCA | 0.931 | 0.788 | 0.624 | 0.622 |

These data show that vessels treated with rapamycin carried by microbubbles have elevated expression of both p21 and p27, the anticipated effect of rapamycin. The 2 mg dose in 35-40 kg pigs is too small for this effect to be due to systemic accumulation of rapamycin at the injured vessel site. This provides evidence that the microbubbles effectively carry the rapamycin to the site of vessel injury and deposit the rapamycin at the injury site.

B. Chronic Effects

The remaining 5 pigs were treated with balloon angioplasty and stent implantation, then divided into (1) control (no drug treatment), (2) rapamycin/PESDA treatment and (3) antisense c-myc/PESDA treatment. Pigs were sacrificed 4 weeks after treatment for analysis of tendency for restenosis. The endpoint for these studies included quantitative angiography and histomorphometry, as described in Materials and Methods below. Histomorphometry data at 28 days post procedure, measured as described in the Examples below, are given in Tables 2 and 3, below.

No evidence of myocardial infarction was seen on gross inspection or after histological evaluation. H&E and VVG-stained sections of all arterial segments were examined. All stents were well developed within the vessel, resulting in thinning of the media adjacent to the stent struts. In the rare vessels with stent protrusion into the adventitia, there was evidence of perivascular hemorrhage. No cases of thrombosis of the treated segment were observed in any of the treatment groups. Complete healing was observed with virtually no toxicity in the treatment groups, and re-endothelialization was complete in all treatment groups.

Neointima from treated arteries was smaller in size than the controls. Control arteries exhibited a substantial neointima, consisting mostly of stellate and spindle-shaped cells, in a loose extracellular matrix. In the antisense treated arteries, the cells of the neointima were morphologically similar to the controls.

Table 2 shows control and rapamycin data for individual vessels. Note that the restenosis process reduces the lumen area and increases the intimal and medial area. Units are in mm and mm².

TABLE 2

| Vessel - Trtmt | Lumen Area | Intimal Area | Medial Area |
|---|---|---|---|
| LAD - rapa 661 | 4.62 ± 1.01 | 3.26 ± 2.18 | 1.52 ± 0.31 |
| LAD - rapa 662 | 8.04 ± 1.59 | 2.94 ± 1.26 | 1.85 ± 0.05 |
| LAD - control | 3.55 ± 0.92 | 2.89 ± 0.93 | 1.43 ± 0.18 |
| RCA - rapa 661 | 7.45 ± 0.32 | 1.64 ± 0.55 | 2.08 ± 0.51 |
| RCA - control | 2.54 ± 1.14 | 6.24 ± 1.15 | 1.87 ± 0.42 |
| LCX - rapa 661 | 2.23 ± 1.57 | 3.53 ± 1.40 | 1.02 ± 0.23 |

Both measurements for LAD lumen area are larger in the rapamycin coated microbubble group than in the control groups (4.62 and 8.04 vs. 3.55), and the RCA lumen area is also much larger than in the control (8.04 vs. 2.54). Although, in this study, the rapamycin treatment did not significantly alter medial area or intimal thickening in the LAD, intimal thickening was greatly reduced in the RCA (1.64 vs. 6.24).

Table 3 shows averaged histomorphometric data from measurements of the individual vessels. For control, n=3; for rapamycin, n=4-6, and for antisense, n=6. Values for the first ten variables (arterial diameter—lumen area) are in mm or mm². Grading systems described by Kornowski et al. and by Suzuki et al. (Circulation 104(10):1188-93, 2001) were used to assess the vessel wall and extent of vascular repair (intimal vascularity; intimal fibrin; intimal SMC content; adventitial fibrosis).

Injury score (IS) and inflammation score were adapted from the scoring system described by Kornowski et al., who observed that implanted stents cause neointimal proliferation proportional to injury. The ratio of neointimal area/injury score (IA/IS) provides a normalized value of intimal area related to the extent of vessel injury.

The values of Intimal Thickness and Intimal Area, as well as the normalized values of IA/IS, show that both therapeutic compositions inhibited stenosis relative to the control, with the rapamycin composition significantly superior to the c-myc composition.

TABLE 3

| | | | c-myc |
|---|---|---|---|
| Variable | Control | Rapamycin | Antisense |
| Arterial Area | 9.70 ± 1.58 | 10.04 ± 2.59 | 10.94 ± 2.09 |
| Intimal Area (IA) | 4.77 ± 1.71 | 1.84 ± 0.44 | 2.83 ± 1.99 |
| Media Area | 1.60 ± 0.24 | 1.62 ± 0.46 | 1.83 ± 0.45 |
| Int/Med Ratio | 3.02 ± 0.80 | 2.11 ± 1.25 | 1.81 ± 1.59 |
| Lumen Area | 3.34 ± 0.72 | 6.55 ± 1.69 | 6.07 ± 3.20 |
| Area % Occl. | 57.53 ± 13.19 | 26.00 ± 19.00 | 33.26 ± 24.63 |
| Lum/Art Ratio | 0.35 ± 0.11 | 0.65 ± 0.16 | 0.55 ± 0.20 |
| Injury Score (IS) | 1.92 ± 0.63 | 1.75 ± 0.46 | 1.13 ± 0.96 |
| IA/IS | 2.48 | 1.05 | 2.50 |
| Inflam Score | 0.67 ± 0.52 | 0.44 ± 0.13 | 0.17 ± 0.30 |
| Intimal Vascularity | 0.42 ± 0.52 | 0.38 ± 0.48 | 0.17 ± 0.30 |
| Intimal Fibrin | 0.17 ± 0.14 | 0.19 ± 0.24 | 0.21 ± 0.25 |
| Intimal SMC Content | 3.00 ± 0.00 | 3.00 ± 0.00 | 3.00 ± 0.00 |
| Adventitial Fibrosis | 1.17 ± 0.76 | 0.88 ± 0.25 | 0.71 ± 0.62 |

IEM = internal elastic lamina; SMC = smooth muscle cell

### EXAMPLES

### Preparation of Albumin-Encapsulated Microbubbles Conjugated to Rapamycin

PESDA (perfluorocarbon-exposed sonicated dextrose/albumin) microbubbles were prepared as described in, for example, U.S. Pat. No. 6,245,747 and PCT Pubn. No. WO 10 2000/02588. In a typical procedure, 5% human serum albumin and 5% dextrose, obtained from commercial sources, were drawn into a 35 mL syringe in a 1:3 ratio, hand agitated with 6-10 mL of decafluorobutane, and sonicated at 20 kilohertz for 75-85 seconds. As described in U.S. Pat. No. 6,245,747, the mean size of four consecutive samples of PESDA microbubbles produced in this manner, as measured with hemocytometry, was 4.6±0.4 microns, and mean concentration, as measured by a Coulter counter, was 1.4×10⁹ bubbles/mL.

A solution of rapamycin in a pharmaceutically acceptable solvent, such as alcohol, DMSO, or castor oil, was incubated with agitation with the PESDA microbubble suspension at room temperature. The mixture was allowed to settle, with the rapamycin-conjugated microbubbles rising to the top. If necessary, the rapamycin solution is sterilized and/or filtered through a micropore filter prior to incubation.

While the invention has been described with reference to specific methods and embodiments, it will be appreciated that various modifications may be made without departing from the invention.

1. A composition comprising:
(i) a suspension of microbubbles which are encapsulated with a filmogenic protein and contain a gas selected from a perfluorocarbon and SF₆, and
(ii) a non-antisense antiproliferative therapeutic agent.

2. The composition of claim 1, wherein the gas is a perfluorocarbon selected from the group consisting of perfluoromethane, perfluoroethane, perfluoropropane, perfluorobutane, and perfluoropentane.

3. The composition of claim 1, wherein the protein is human serum albumin.

4. The composition of claim 1, wherein the agent is selected from the group consisting of rapamycin, paclitaxel, docetaxel, tacrolimus, and active analogs, derivatives or prodrugs of these compounds.

5. The composition of claim 5, wherein the agent is selected from the group consisting of rapamycin, paclitaxel, and docetaxel.

6. The composition of claim 1, wherein the agent is selected from the group consisting of cisplatin, carboplatin, etoposide, tamoxifen, methotrexate, 5-fluorouracil, adriamycin, daunorubicin, doxorubicin, amsacrine, mitotane, topotecan, tretinoin, hydroxyurea, procarbazine, carmustine, mechlorethamine hydrochloride, cyclophosphamide, ifosfamide, chlorambucil, melphalan, busulfan, thiotepa, carmustine, estramustine, dacarbazine, omustine, streptozocin, vincristine, vinblastine, vinorelbine, vindesine, fludarabine, fluorodeoxyuridine, cytosine arabinoside, cytarabine, azidothymidine, cysteine arabinoside, azacytidine, mercaptopurine, thioguanine, cladribine, pentostatin, arabinosyl, adenine, dactinomycin, daunorubicin, doxorubicin, amsacrine, idarubicin, mitoxantrone, bleomycin, plicamycin, ansamitomycin, mitomycin, aminoglutethimide, and flutamide.

7. The composition of claim 6, wherein the agent is selected from the group consisting of cisplatin, carboplatin, etoposide, tamoxifen, methotrexate, 5-fluorouracil, adriamycin, daunorubicin, doxorubicin, vincristine, and vinblastine.

8. The composition of claim 1, wherein said composition is formed by incubating said agent with said suspension of microbubbles.

9. A method for delivering an antiproliferative therapeutic agent to the site of a tumor in a subject, comprising:
administering parenterally to a subject having said tumor a composition comprising said agent and a suspension of microbubbles which are encapsulated with a filmogenic protein and contain a gas selected from a perfluorocarbon and SF₆.

10. The method of claim 9, wherein said administration is carried out without application of external stimulation to said composition during or following administration.

11. The method of claim 9, wherein the gas is a perfluorocarbon selected from the group consisting of perfluoromethane, perfluoroethane, perfluoropropane, perfluorobutane, and perfluoropentane. albumin.

12. The method of claim 9, wherein the protein is human serum

13. The method of claim 9, wherein the agent is selected from the group consisting of rapamycin, paclitaxel, docetaxel, tacrolimus, and active analogs, derivatives or prodrugs of these compounds.

14. The method of claim 13, wherein the agent is selected from the group consisting of rapamycin, paclitaxel, and docetaxel.

15. The method of claim 9, wherein the agent is selected from the group consisting of cisplatin, carboplatin, etoposide, tamoxifen, methotrexate, 5-fluorouracil, adriamycin, daunorubicin, doxorubicin, amsacrine, mitotane, topotecan, tretinoin, hydroxyurea, procarbazine, carmustine, mechlorethamine hydrochloride, cyclophosphamide, ifosfamide, chlorambucil, melphalan, busulfan, thiotepa, carmustine, estramustine, dacarbazine, omustine, streptozocin, vincristine, vinblastine, vinorelbine, vindesine, fludarabine, fluorodeoxyuridine, cytosine arabinoside, cytarabine, azidothymidine, cysteine arabinoside, azacytidine, mercaptopurine, thioguanine, cladribine, pentostatin, arabinosyl adenine, dactinomycin, daunorubicin, doxorubicin, amsacrine, idarubicin, mitoxantrone, bleomycin, plicamycin, ansamitomycin, mitomycin, aminoglutethimide, and flutamide.

16. The method of claim 15, wherein the antiproliferative agent is selected from the group consisting of cisplatin, carboplatin, etoposide, tamoxifen, methotrexate, 5-fluorouracil, adriamycin, daunorubicin, doxorubicin, vincristine, and vinblastine.

17. The method of claim 9, wherein the agent is a non-antisense agent.

18. The method of claim 9, wherein said composition is formed by incubating said agent with said suspension of microbubbles.
